(19) United States
(12) Patent Application Publication
(10) Pub. No.: US 2016/0180021 A1
(43) Pub. Date: Jun. 23, 2016

(54) X-RAY DIAGNOSTIC IMAGING APPARATUS AND CONTROL METHOD THEREFOR

(71) Applicant: CANON KABUSHIKI KAISHA, Tokyo (JP)

(72) Inventor: Miho Higano, Yokohama-shi (JP)

(21) Appl. No.: 14/966,022

(22) Filed: Dec. 11, 2015

(30) Foreign Application Priority Data
Dec. 22, 2014 (JP) 2014-259277

(51) Int. Cl. G06F 19/00; G06F 17/30; A61B 6/00

(52) U.S. Cl. CPC G06F 19/321; A61B 6/5294; G06F 17/30265; G06F 17/3028; G06F 17/30864; G06F 19/3443

(57) ABSTRACT

This invention can present failure imaging caused by set imaging information or reduce the occurrence of imaging failures more than the related art. An apparatus according to the invention includes a first searching unit configured to search, when a reception unit receives new imaging information for newly performing imaging, past imaging information stored in a storage unit for imaging information which has a similarity degree not less than a preset threshold relative to the new imaging information and is assigned with an evaluation result indicating an imaging failure, and a second searching unit configured to search imaging information corrected with respect to the imaging information found by the first searching unit for imaging information assigned with the evaluation result indicating an imaging success as recommended imaging information in place of the new imaging information.

### BACKGROUND OF THE INVENTION

1. Field of the Invention

The present invention relates to an x-ray diagnostic imaging apparatus and a control method therefor.

2. Description of the Related Art

Recently, in medical sites, x-ray diagnostic imaging apparatuses are often used. When acquiring x-ray image data by imaging an object by using such an x-ray diagnostic imaging apparatus, imaging may fail due to x-ray imaging conditions. As a technique associated with imaging failures by such x-ray diagnostic imaging apparatuses, the technique disclosed in Japanese Patent Laid-Open No. 2009-268586 is known.

According to this literature, imaging conditions set when imaging failure occurred in the past are stored. If there are imaging conditions which are set at the time of the occurrence of a past imaging failure and are similar to imaging conditions input when imaging an object, a corresponding warning is displayed. This allows the user to know in advance that input imaging conditions may cause an imaging failure, before executing imaging. It can therefore be expected that imaging failures will be suppressed.

The above literature, however, only indicates that current imaging conditions, without any change, are likely to lead to an imaging failure. Even if the imaging conditions are changed, there is still a possibility that the imaging may fail. That is, there is still room for improvement.

### SUMMARY OF THE INVENTION

The present invention has been made in consideration of the above problem and provides a technique of actively reducing imaging failures.

According to an aspect of the invention, there is provided a diagnostic imaging apparatus including an x-ray imaging unit, the apparatus comprising: a reception unit configured to receive imaging information constituted by parameters concerning a target person and parameters concerning conditions for obtaining an x-ray image by making the x-ray imaging unit perform imaging; a storage unit configured to store an evaluation result concerning an x-ray image obtained by making the x-ray imaging unit perform imaging in accordance with the imaging information in association with the imaging information; a first searching unit configured to search, when the reception unit receives new imaging information for newly performing imaging, past imaging information stored in the storage unit for imaging information which has a similarity degree not less than a preset threshold relative to the new imaging information and is assigned with an evaluation result indicating an imaging failure; and a second searching unit configured to search imaging information corrected with respect to the imaging information found by the first searching unit for imaging information assigned with the evaluation result indicating an imaging success as recommended imaging information in place of the new imaging information.

According to the present invention, it is possible to prevent an imaging failure caused by set imaging information or reduce the occurrence of imaging failures as compared with the related art.

Further features of the present invention will become apparent from the following description of exemplary embodiments (with reference to the attached drawings).

### BRIEF DESCRIPTION OF THE DRAWINGS

The accompanying drawings, which are incorporated in and constitute a part of the specification, illustrate embodiments of the invention and, together with the description, serve to explain the principles of the invention.

FIG. 1 is a block diagram showing the arrangement of an x-ray diagnostic imaging apparatus;

FIG. 2 is a block diagram showing the functional arrangement of the x-ray diagnostic imaging apparatus;

FIG. 3 is a view showing an example of the data configuration of a medical treatment DB;

FIG. 4 is a flowchart showing processing performed by the x-ray diagnostic imaging apparatus;

FIG. 5 is a flowchart showing processing performed by the x-ray diagnostic imaging apparatus;

FIG. 6 is a flowchart showing processing performed by the x-ray diagnostic imaging apparatus; and

FIG. 7 is a table showing an example of the data configuration of a medical treatment DB.

### DESCRIPTION OF THE EMBODIMENTS

The embodiments of the present invention will be described below with reference to the accompanying drawings. Note that each embodiment will exemplify an apparatus, as an x-ray diagnostic imaging apparatus, which includes an x-ray tube and a flat panel detector which generates an electrical signal by detecting x-rays generated by the x-ray tube and transmitted through an object. Note however that this apparatus may be an x-ray CT apparatus or the like, and the present invention is not limited by the type of apparatus.

### First Embodiment

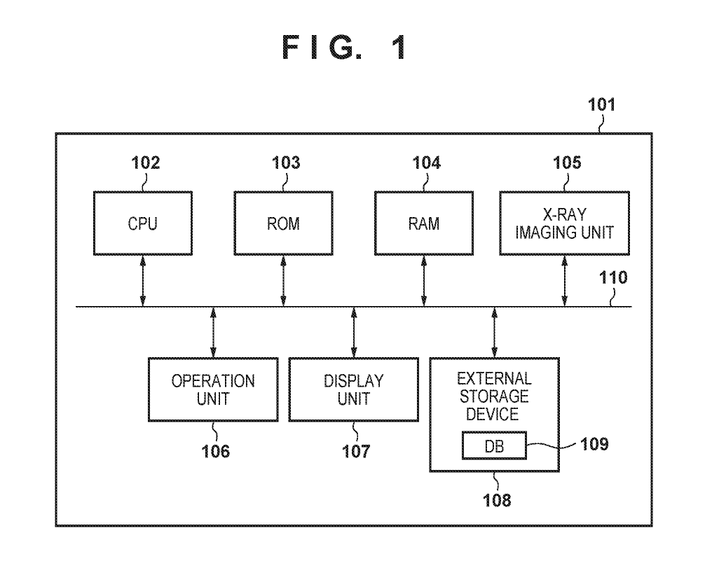

FIG. 1 is a block diagram showing the arrangement of an x-ray diagnostic imaging apparatus 101 according to the first embodiment. The apparatus 101 includes a CPU 102 which controls the overall apparatus, a ROM 103 storing a boot program, a BIOS, and the like, and a RAM 104. The RAM 104 is used to store programs to be executed by the CPU 102 and temporarily store obtained x-ray images and the like. The RAM 104 is also used as a work area for the CPU 102. The apparatus 101 also includes an x-ray imaging unit 105 including an x-ray tube, a flat panel detector, and a driving unit which drives them. The apparatus 101 includes an operation unit 106 for receiving an instruction from the operator and a display unit 107 for displaying obtained images, various types of messages, and the like. In addition, the apparatus 101 includes an external storage device 108 typified by a hard disk which is used to store an OS (Operating System), various types of program files, and files concerning information about objects and x-ray images obtained by imaging. A database (DB) 109 (to be described later) is stored in the external storage device 108. These constituent elements are connected to a bus 110. This allows the CPU 102 to control the respective constituent elements. Although the external storage device 108 has been described as a hard disk drive, the type of storage device to be used is not specifically limited. For example, this storage device may be a memory card, detachable FD (Flexible Disk), optical disk such as CD (Compact Disk), magnetic or optical card, IC card, or memory card. The x-ray diagnostic imaging apparatus 101 may also include a network interface (not shown) for connection to a network such as the Internet. In this case, the DB 109 may hold another apparatus (server) on the network. When the server on the network holds the DB 109, the apparatus 101 transmits a search request to the server and receives a search result from the server. In the following description, assume that the apparatus 101 holds the DB 109.

In the above arrangement, when the power supply of this apparatus is turned on, the CPU 102 executes the boot program in the ROM 103. The CPU 102 loads the OS stored in the external storage device 108 into the RAM 104. The CPU 102 then loads a program functioning as the x-ray diagnostic imaging apparatus into the RAM 104 and executes the program. As a result, the apparatus 101 functions as an x-ray diagnostic imaging apparatus.

The DB 109 stores various types of information concerning x-ray imaging. The DB 109 stores, for example, information concerning x-ray imaging, including patient information, medical treatment information, imaging conditions, imaging result information, and re-imaging information. In this case, patient information is information concerning a patient (target person), for example, the identification information, name, age, sex, height, and weight of the patient. Medical treatment information is information concerning a disease name and a treatment which has been applied to or will be applied to the patient. Imaging conditions are information to be used at the time of x-ray imaging, including a body part to be imaged, an imaging posture, x-ray generation conditions (the tube current and tube voltage of the x-ray tube), sensor conditions (for example, sensitivity), and image processing conditions (for example, the type of filter for image processing). Imaging result information is information concerning an imaging result, for example, information indicating whether imaging has succeeded or failed. Re-imaging information is information concerning re-imaging, including, for example, the identification information of an imaging case having undergone re-imaging and imaging conditions at the time of re-imaging. Note that a patient DB may be separately provided. This DB is used to receive the input of a patient ID, and patient information is acquired from the patient ID.

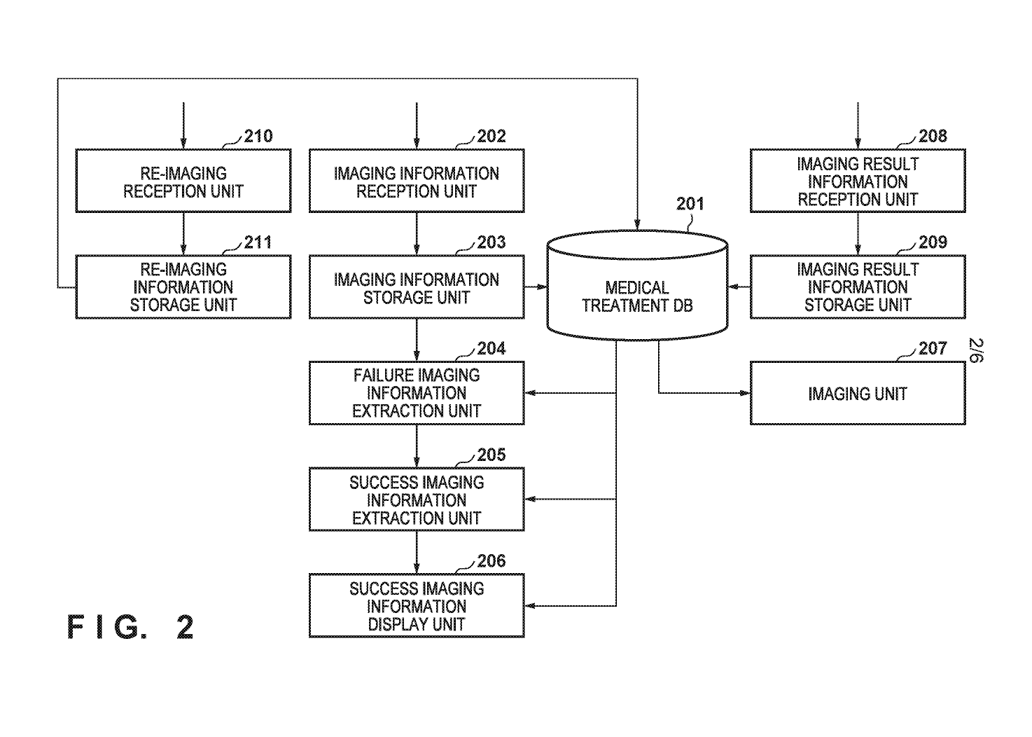

FIG. 2 is a block diagram showing the functional arrangement of the x-ray diagnostic imaging apparatus 101 according to the embodiment. FIG. 2 corresponds to an arrangement in which the CPU 102 in the arrangement in FIG. 1 loads programs associated with the x-ray diagnostic imaging apparatus from the external storage device 108 into the RAM 104 and executes the programs. Therefore, some of the constituent elements in FIG. 2 are implemented by the CPU 102. This apparatus includes a medical treatment DB 201. The medical treatment DB 201 corresponds to the DB 109 and stores patient information associated with x-ray imaging, medical treatment information, imaging conditions, imaging result information, and re-imaging information. Note that imaging conditions may include an x-ray diagnostic imaging apparatus type and a sensor type.

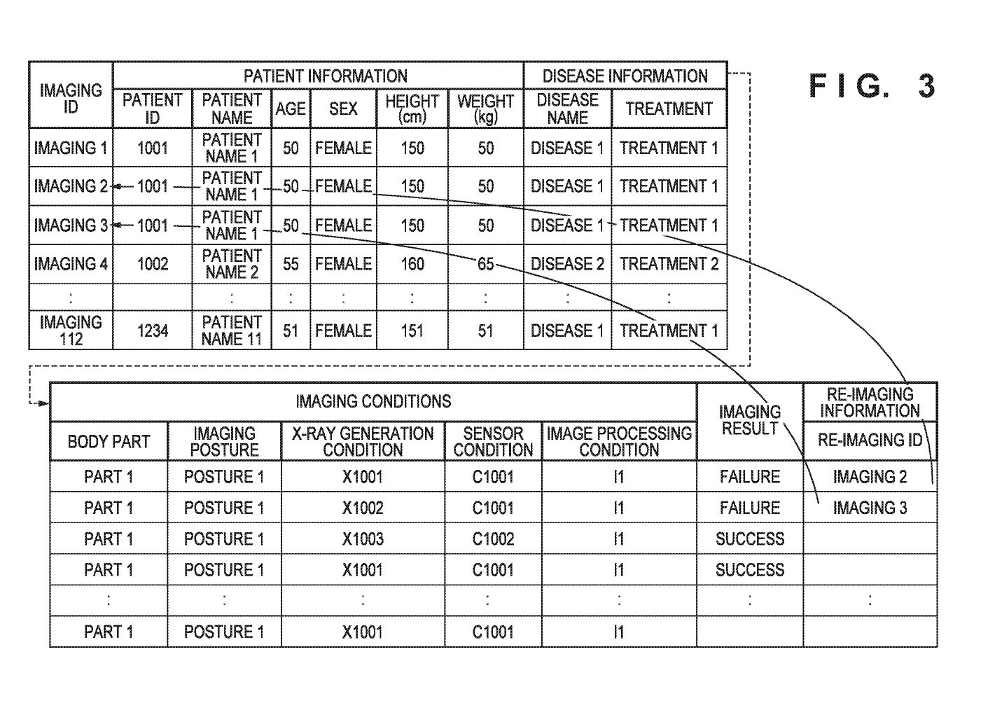

FIG. 3 is a view showing an example of the data arrangement of the medical treatment DB 201. Due to space limitation, FIG. 3 shows this data arrangement in the form of two tables. In practice, however, the data arrangement has a single table form, with the two tables being connected to each other as indicated by the broken line. The medical treatment DB 201 includes an imaging ID item, a patient information item, a disease information item, an imaging condition item, an imaging result item, and re-imaging information item.

The imaging ID item includes fields for storing identification information of imaging cases. The patient information item includes patient ID, patient name, age, sex, height (cm), and weight (kg) fields. A patient ID links to a patient DB (not shown) and serves to uniquely identify the patient. The disease information item includes disease name and treatment fields. The imaging condition item includes body part to be imaged, imaging posture, x-ray generation condition, sensor condition, and image processing condition fields. The imaging result information item includes fields for storing imaging results. An imaging result is evaluation result information indicating whether imaging has succeeded or failed. The re-imaging information item includes a re-imaging ID item. The re-imaging ID item includes fields each for storing an imaging ID set when re-imaging is performed.

Although according to the above description, the data arrangement explicitly includes imaging result information, it is possible to determine an image result depending on the presence/absence of re-imaging information. For example, if an imaging ID is set in a re-imaging information field, the imaging result may be regarded as “failure”. If no re-imaging ID is set in the re-imaging information field, the imaging result may be regarded as “success”. Alternatively, if there is an imaging case with the same patient ID within a predetermined period, the imaging result may be regarded as “failure”. If there is no imaging case with the same patient ID within a predetermined period, the imaging result may be regarded as “success”.

Note that the following cases will be considered as easy cases which cause imaging failures: a case in which imaging was performed when an x-ray tube current (one of x-ray generation conditions) for a thin patient was set in spite of the fact that the examinee is an obese person; and a case in which a chest region which can be imaged with a low dose was set in spite of the fact that an abdominal region was actually imaged.

Patient information, disease information, and imaging conditions are stored in the DB 201 by being input by an operator (a health professional such as a doctor or technician) via the operation unit 106 before the execution of x-ray imaging. Failure information and re-imaging information are stored in the DB 201 by being input by the operator via the operation unit 106 after the execution of x-ray imaging. Assume that x-ray imaging has been actually completed, and the display unit 107 displays the obtained image. In this case, when the operator determines that the result has not reached an intended quality and inputs information indicating “failure” via the operation unit 106, failure information is stored in the DB 201. When the operator inputs an instruction to re-image via the operation unit 106, next “imaging ID” is stored in the “re-imaging information” field to perform a re-imaging operation. Assume that when having input an instruction to re-image, the operator copies and pastes already input information as patient information and disease information to allow him/her to perform only the operation of re-setting imaging conditions associated with re-imaging.

Alternatively, patient information, disease information, and imaging conditions, which are obtained before x-ray imaging, and failure information and re-imaging information, which are obtained after x-ray imaging, may be separately stored in the DB 201, and information for associating them with each other may be provided. As described above, the data arrangement is not limited to that in this embodiment as long as patient information, disease information, and imaging conditions are associated with failure information and re-imaging information, which are obtained after x-ray imaging.

The DB 201 in FIG. 3 therefore indicates that imaging indicated by the imaging ID “imaging 1” (first record) has failed, and re-imaging (its imaging ID is “imaging 2”) has been performed. The DB 201 indicates that re-imaging has been performed upon correcting (changing) an x-ray generation condition of imaging conditions from X1001 to X1002 without changing the remaining pieces of imaging information after correction, and the re-imaging result also indicates a failure. The DB 201 also indicates that re-re-imaging (its imaging ID is “imaging 3”) has been performed in accordance with corrected imaging information upon correcting (changing) an x-ray generation condition of the imaging conditions from X1002 to X1003 and a sensor condition from C1001 to C1002, and the imaging result indicates a success.

Referring back to FIG. 2, an imaging information reception unit 202 receives information concerning x-ray imaging which is input by the operator via the operation unit 106. The imaging information reception unit 202 receives, for example, new imaging information such as patient information, disease information, and imaging conditions. When performing re-imaging, the imaging information reception unit 202 further receives various types of information newly input by the operator. An imaging information storage unit 203 stores the patient information, the medical treatment information, and the imaging conditions, received by the imaging information reception unit 202, in the DB 201 in association with each other.

For example, in association with the imaging ID “imaging 1” input by the operator, the imaging information storage unit 203 receives the patient ID “1001”, patient name “patient name 1”, age “50”, sex “female”, height “150 (cm)”, weight “50 (kg)”, disease name “disease 1”, treatment “treatment 1”, body part to be imaged “part 1”, imaging posture “posture 1”, x-ray generation condition “X1001”, sensor condition “C1001”, and image processing condition “I1”, and stores them in the DB 201 shown in FIG. 3. In addition, assume that after an image obtained by imaging was displayed on the display unit 107, the imaging information reception unit 202 has received “failure” and an instruction to execute “re-imaging” from the operator. In this case, the imaging information storage unit 203 stores “failure” in the imaging result field and “imaging 2” in the re-imaging ID field. The imaging information storage unit 203 then newly registers “imaging 2” in the imaging ID field, and copies and pastes, as patient information and disease information, pieces of corresponding information associated with the imaging ID “imaging 1”. The imaging information reception unit 202 then receives the re-settings of the imaging conditions associated with re-imaging.

Note that in the following description, information corresponding to one record (one row) shown in FIG. 3 will be referred to as “imaging information”. In addition, imaging information corresponding to an “imaging result” field in which “failure” is set will be referred to as “failure imaging information”, and imaging information corresponding to an “imaging result” field in which “success” is set will be referred to as “success imaging information”.

A failure imaging information extraction unit 204 compares imaging information received by the imaging information reception unit 202 with the imaging conditions of past failure imaging information stored in the medical treatment DB 201, and extracts (searches for) the imaging information of past failure imaging which exhibits a difference equal to or less than a preset threshold.

A success imaging information extraction unit 205 extracts (or searches for) success imaging information corresponding to the imaging result field in which “success” is set in accordance with the re-imaging ID associated with the past failure imaging extracted by the failure imaging information extraction unit 204.

Assume that in the medical treatment DB 201 shown in FIG. 3, when newly performing x-ray imaging, the imaging information reception unit 202 has received the imaging ID “imaging 112”, the patient ID “1234”, the patient name “patient name 11”, the age “51”, the sex “female”, the height “151 (cm)”, the weight “51 (kg)”, the disease name “disease 1”, the treatment “treatment 1”, the body part to be imaged “part 1”, the imaging posture “posture 1”, the x-ray generation condition “X1001”, and the sensor condition “C1001”. In this case, the failure imaging information extraction unit 204 extracts, from past failure imaging information stored in the DB 201, “failure imaging information” whose similarity degree (similarity) with respect to imaging information corresponding to “imaging ID 112” received to perform the current imaging is equal to or more than a preset threshold.

In this case, that the similarity degree between two pieces of imaging information is high is equivalent to that the distance between multidimensional coordinate spaces respectively indicated by the numbers of parameters constituting the two pieces of imaging information is small. Therefore, the processing of extracting failure imaging information B having a similarity degree equal to or more than a preset threshold with respect to imaging information A can be expressed in another way as the processing of extracting failure imaging information B located at a distance from a coordinate position specified by the parameters constituting imaging information A by a predetermined threshold or less.

Parameters (can be expressed as a coordinate axis) representing a distance in this embodiment are information which can influence imaging and include, in the embodiment, 11 pieces of information including an age, sex, height, weight, disease name, treatment, body part to be imaged, imaging posture, x-ray generation condition, sensor condition, and image processing condition. That is, the imaging information can be said to be 11-dimensional information. Note however that some of these pieces of information are parameters which cannot be directly numerically expressed, and hence are converted into numerical values by referring to a table prepared in advance. For example, “sex” is numerically expressed based on a separate dedicated table such that male and female are respectively converted into 0 and 1.

In this case, the types of parameters are represented by P1, P2, . . . , Pn (n=11 in this embodiment). Assume that parameter values corresponding to the imaging ID “imaging 112” received by the imaging information reception unit 202 to currently and newly perform imaging are represented by P1(x), P2(x), . . . , Pn(x). In addition, assume that the parameter values of past failure imaging information in the DB 201 are represented by P1(i), P2(i), . . . , Pn(i). In this case, a distance L between the two pieces of imaging information is given by

L={α1×(P1(x)−P1(i))²+α2×(P2(x)−P2(i))²+ . . . +αn×(Pn(x)−Pn(i))²}^{1/2}

where α1, α2, . . . , αn are weight coefficients set in advance for the respective types of parameters. Note that the sum of squares of differences may be simply calculated according to the following equation because it is only required to determine the magnitude of a distance and it is preferable to simplify computation.

L=α1×(P1(x)−P1(i))²+α2×(P2(x)−P2(i))²+ . . . +αn×(Pn(x)−Pn(i))²

The failure imaging information extraction unit 204 extracts and outputs failure imaging information specified by i corresponding to the shortest distance among distances L equal to or less than a predetermined threshold. Note that if no failure imaging information is found which has the distance L equal to or less than the threshold, the failure imaging information extraction unit 204 outputs information indicating that no appropriate failure imaging information currently exists.

Assume that the imaging information reception unit 202 has received imaging information associated with the imaging ID “imaging 112” in FIG. 3, and the failure imaging information extraction unit 204 has extracted imaging information corresponding to the imaging ID “imaging 1” exhibiting the smallest distance L equal to or less than the threshold among imaging information corresponding to the imaging result “failure”.

The success imaging information extraction unit 205 analyzes the imaging information corresponding to the imaging ID “imaging 1”, and determines from the corresponding re-imaging information that imaging corresponding to the imaging ID “imaging 2” has been performed again. The success imaging information extraction unit 205 therefore analyzes imaging information corresponding to the imaging ID “imaging 2”. As a result of this analysis, the success imaging information extraction unit 205 determines that “imaging 2” has failed, and re-re-imaging corresponding to “imaging 3” has been performed. The success imaging information extraction unit 205 therefore analyzes imaging information corresponding to the imaging ID “imaging 3”. As a result of this analysis, the success imaging information extraction unit 205 determines that “imaging 3” has succeeded, and hence decides imaging information corresponding to the imaging ID “imaging 3” as success imaging information.

A success imaging information display unit 206 performs following processes (1) and (2):

(1) When the failure imaging information extraction unit 204 was able to extract imaging information specified by “i” corresponding to the distance L equal to or less than the threshold, the success imaging information display unit 206 displays, on the display unit 107, a message indicating that the received imaging information may be inappropriate.

(2) The success imaging information display unit 206 displays, on the display unit 107, imaging conditions associated with success imaging and extracted by the success imaging information extraction unit 205 as recommended imaging conditions to prompt to change the imaging conditions.

Upon seeing the recommended imaging conditions, the operator changes the x-ray generation condition in the imaging conditions corresponding to the imaging ID “imaging 112” from “X1001” to “X1003”, and the sensor condition from “C1001” to “C1002”, and issues an instruction to perform x-ray imaging via the operation unit 106. If this imaging has succeeded, the imaging result corresponding to the imaging ID “imaging 112” of interest is naturally “success”. Note that when performing display based on the process (2), all the imaging conditions corresponding to the imaging ID “imaging 112” may be replaced by information corresponding to success imaging in accordance with the operation of the user on a predetermined button or key.

An imaging unit 207 performs x-ray imaging in accordance with imaging information stored in the medical treatment DB 201. An imaging result information reception unit 208 receives an imaging ID and an imaging result input by the operator. An imaging result information storage unit 209 stores the imaging ID and the imaging result received by the imaging result information reception unit 208 in the medical treatment DB 201 in association with each other.

The medical treatment DB 201 shown in FIG. 3 receives, for example, the imaging ID “imaging 1” and the imaging result “failure” and stores the imaging ID and the imaging result in association with each other. A re-imaging reception unit 210 receives the designation of re-imaging input by the operator.

A re-imaging information storage unit 211 stores a newly issued imaging ID for re-imaging in the medical treatment DB 201 in association with target re-imaging designated by the re-imaging reception unit 210. When the medical treatment DB 201 shown in FIG. 3 receives, for example, an instruction to perform re-imaging corresponding to the imaging ID “imaging 1” from the operator, the imaging ID “imaging 2” is newly issued. The re-imaging information storage unit 211 stores “imaging 2” as a re-imaging ID corresponding to the imaging ID “imaging 1” in the medical treatment DB 201 in associating with each other.

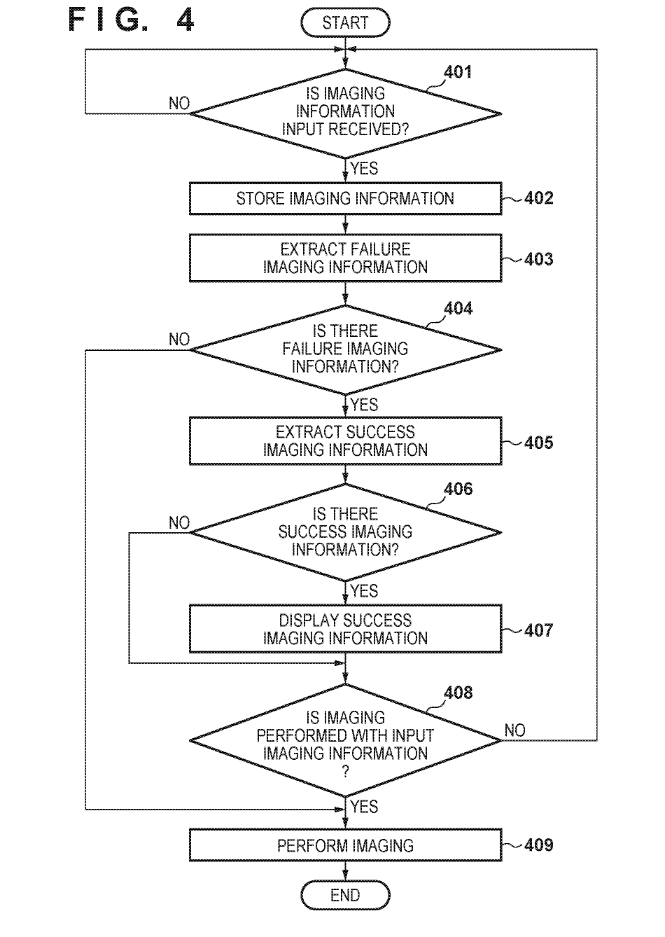

FIG. 4 is a flowchart showing imaging processing starting from imaging information input processing by the x-ray diagnostic imaging apparatus 101.

In step 401, the imaging information reception unit 202 receives the input of new imaging information (imaging information input processing). The process then advances to step 402. In step 402, the imaging information storage unit 203 stores the newly received imaging information in the medical treatment DB 201 (imaging information storage processing).

In step 403, the failure imaging information extraction unit 204 obtains distances L between imaging conditions for the new imaging information received by the imaging information reception unit 202 and imaging conditions of past failure imaging information stored in the medical treatment DB 201 by the imaging information storage unit 203, and extracts failure imaging information exhibiting the minimum length L equal to or less than a threshold (failure imaging information extraction processing).

In step 404, the failure imaging information extraction unit 204 determines whether failure imaging information has been extracted in step 403. If YES in step 404, the process advances to step 405. If the failure imaging information extraction unit 204 determines that no failure imaging information has been extracted in step 403, the process advances to step 409.

In step 405, the success imaging information extraction unit 205 extracts re-imaging information from imaging IDs stored in re-imaging information associated with the failure imaging information extracted by the failure imaging information extraction unit 204 (re-imaging information extraction means). The success imaging information extraction unit 205 further extracts success imaging information corresponding to extracted re-imaging information exhibiting the imaging result “success”. If the imaging result of the re-imaging information is “failure”, the success imaging information extraction unit 205 repeats re-imaging information extraction until it extracts re-imaging information corresponding to the imaging result “success” or extracts no re-imaging information (success imaging information extraction processing).

In step 406, the success imaging information extraction unit 205 determines whether success imaging information was able to be extracted. If YES in step 406, the process advances to step 407. If the success imaging information extraction unit 205 has extracted no success imaging information in step 405, the process advances to step 408.

In step 407, the success imaging information display unit 206 displays the imaging conditions for the success imaging information extracted by the success imaging information extraction unit 205 as recommended imaging conditions, together with a message indicating that the imaging conditions for the imaging information received by the imaging information reception unit 202 may be inappropriate (success imaging information display processing).

In step 408, the success imaging information display unit 206 receives an input indicating whether to continue imaging with the imaging information received by the imaging information reception unit 202. If the input indicates that imaging is to be continued, the process advances to step 409. In contrast to this, if the success imaging information display unit 206 has received an input indicating that imaging is not continued with the imaging information received by the imaging information reception unit 202, the process returns to step 401.

In step 409, the imaging unit 207 (corresponding to the x-ray imaging unit 105 in FIG. 1) performs imaging in accordance with the imaging information received by the imaging information reception unit 202 (imaging processing). An x-ray image obtained by imaging is stored in the external storage device 108 in association with the patient ID. With the above operation, the processing is complete.

Note that according to the above description, only when both failure imaging information and success imaging information are extracted, information indicating a possibility that set imaging conditions are inappropriate is displayed in step 407. However, when failure imaging information is extracted, information indicating a possibility that set imaging conditions are inappropriate may be displayed.

Specific processing to be performed when the imaging information reception unit 202 has newly received the input of imaging information including the imaging ID “imaging 112” will be subsequently described with reference to FIG. 3. In this case, in step 402, the imaging information storage unit 203 stores the imaging information including the imaging ID “imaging 112” in the medical treatment DB 201. In the case shown in FIG. 3, the following pieces of information are stored in the medical treatment DB in association with each other: the imaging ID “imaging 112”, patient ID “1234”, patient name “patient name 11”, age “51”, sex “female”, height “151 (cm)”, weight “51 (kg)”, disease name “disease 1”, treatment “treatment 1”, body part to be imaged “part 1”, imaging posture “posture 1”, x-ray generation condition “X1001”, sensor condition “C1001”, and image processing condition “I1”.

In step 403, the failure imaging information extraction unit 204 extracts failure imaging information exhibiting the minimum distance equal to or less than a preset threshold from past failure imaging information stored in the medical treatment DB 201 by the imaging information storage unit 203 with respect to the imaging conditions for the new imaging information received by the imaging information reception unit 202.

Assume that, in accordance with FIG. 3, the failure imaging information extraction unit 204 has extracted imaging information corresponding to the imaging ID “imaging 1” exhibiting the minimum distance equal to or less than the threshold with respect to the imaging information corresponding to the imaging ID “imaging 112”. That is, assume that the imaging ID “imaging 112” and “imaging 1” differ in only the following types of parameters: “age” (difference is 1 (year)); “height” (difference is 1 (cm)); and “weight” (difference is 1 (kg)), and are the same in the remaining types of parameters, and hence there are no other pieces of information which exhibit smaller differences.

The failure imaging information extraction unit 204 therefore extracts, as past failure imaging information corresponding to the imaging ID “imaging 112”, imaging information associated with the imaging ID “imaging 1”, that is, the imaging ID “imaging 1”, patient ID “1001”, patient name “patient name 1”, age “50”, sex “female”, height “150 (cm)”, weight “50 (kg)”, disease name “disease 1”, treatment “treatment 1”, body part to be imaged “part 1”, imaging posture “posture 1”, x-ray generation condition “X1001”, sensor condition “C1001”, image processing condition “I1”, imaging result “failure”, and re-imaging ID “imaging 2”.

In step 405, the success imaging information extraction unit 205 extracts re-imaging information in accordance with the re-imaging ID associated with the failure imaging information extracted by the failure imaging information extraction unit 204, and further extracts success imaging information in accordance with the imaging result of the re-imaging information.

In the case shown in FIG. 3, imaging information corresponding to the imaging ID “imaging 2” is obtained in accordance with the re-imaging ID “imaging 2” associated with the imaging ID “imaging 1”. With this imaging information, the following information is obtained: the imaging ID “imaging 2”, patient ID “1001”, patient name “patient name 1”, age “50”, sex “female”, height “150 (cm)”, weight “50 (kg)”, disease name “disease 1”, treatment “treatment 1”, imaging posture “posture 1”, body part to be imaged “part 1”, x-ray generation condition “X1002”, sensor condition “C1001”, image processing condition “I1”, imaging result “failure”, and re-imaging ID “imaging 3”. However, since “failure” is set in the imaging result field of this imaging information, and “imaging 3” is set in the re-imaging ID field, it is obvious that re-imaging has been performed as “imaging 3”. Therefore, the success imaging information extraction unit 205 extracts imaging information corresponding to the imaging ID “imaging 3”. This imaging information includes the imaging ID “imaging 3”, patient ID “1001”, patient name “patient name 1”, age “50”, sex “female”, height “150 (cm)”, weight “50 (kg)”, disease name “disease 1”, treatment “treatment 1”, body part to be imaged “part 1”, imaging posture “posture 1”, x-ray generation condition “X1003”, sensor condition “C1002”, image processing condition “I1”, and imaging result “success”. Since the imaging result is “success”, the success imaging information extraction unit 205 extracts imaging information corresponding to the imaging ID “imaging 3” as success imaging information.

In step 407, the success imaging information display unit 206 displays, as recommended imaging conditions, the imaging conditions for success imaging information extracted by the success imaging information extraction unit 205, together with a message indicating that the imaging conditions for the imaging information received by the imaging information reception unit 202 may be inappropriate.

In the case shown in FIG. 3, the success imaging information display unit 206 displays the above information together with a message indicating that the imaging conditions for imaging information received by the imaging information reception unit 202 and associated with the imaging ID “imaging 112”, that is, the body part to be imaged “part 1”, imaging posture “posture 1”, x-ray generation condition “X1001”, sensor condition “C1001”, and image processing condition “I1” may be inappropriate. In addition, the success imaging information display unit 206 displays, as recommended imaging conditions, the imaging conditions for the imaging information associated with the imaging ID “imaging 3” extracted by the success imaging information extraction unit 205, that is, the body part to be imaged “part 1”, imaging posture “posture 1”, x-ray generation condition “X1003”, sensor condition “C1002”, and image processing condition “I1”. Note that the success imaging information display unit 206 may display identical information and different information of the imaging conditions received by the imaging information reception unit 202 and the imaging conditions extracted by the success imaging information extraction unit 205 in different forms (for example, in blue for identical items and in red for different items).

In step 408, the success imaging information display unit 206 receives, via the operation unit 106, an input indicating whether to continue imaging with the imaging information received by the imaging information reception unit 202. When continuing imaging, the process advances to step 409. If the success imaging information display unit 206 does not continue imaging with the imaging condition received by the imaging information reception unit 202, the process advances to step 401.

In the case shown in FIG. 3, if the success imaging information display unit 206 receives, in step 408, the input indicating that imaging is continued with the imaging information received by the imaging information reception unit 202, that is, the imaging information associated with the imaging ID “imaging 112”, the process advances to step 409, in which imaging processing is performed with the imaging information associated with the imaging ID “imaging 112”. If the operator has changed the imaging conditions based on recommended imaging conditions, imaging is performed with the following information: the imaging ID “imaging 112”, the patient ID “1234”, the patient name “patient name 11”, the age “51”, the sex “female”, the height “151 (cm)”, the weight “50 (kg)”, the disease name “disease 1”, the treatment “treatment 1”, the body part to be imaged “part 1”, the imaging posture “posture 1”, the x-ray generation condition “X1003”, the sensor condition “C1002”, and image processing condition “I1”.

In contrast, if the success imaging information display unit 206 receives the input indicating that imaging is not continued with the imaging information received by the imaging information reception unit 202, that is, the imaging information associated with the imaging ID “imaging 112”, the process advances to step 401, in which the input of imaging information from the operator is received again.

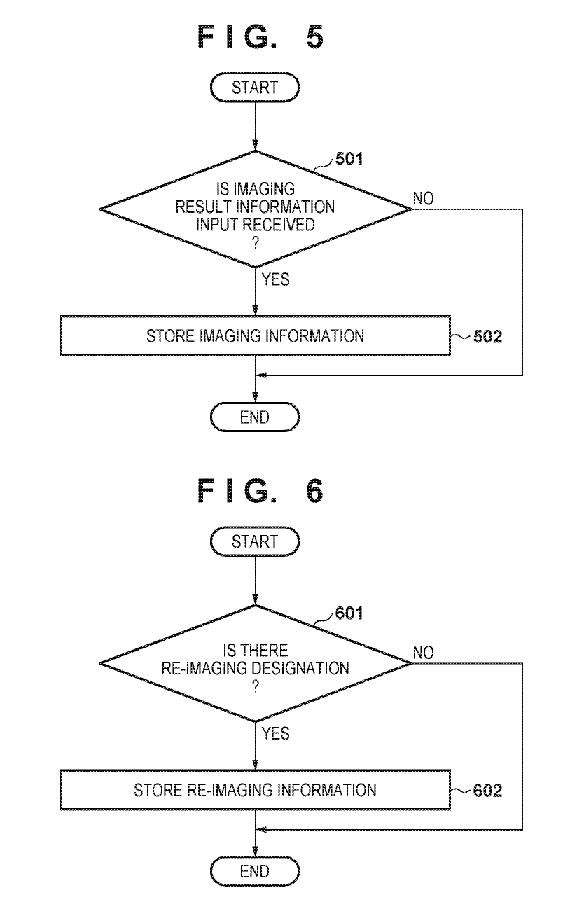

FIG. 5 is a flowchart showing result input processing to be performed after an image obtained by imaging processing by the x-ray diagnostic imaging apparatus 101 is displayed on the display unit 107.

If the imaging result information reception unit 208 receives the input of new imaging result information in step 501 (imaging result information input processing), the process advances to step 502. In step 502, the imaging result information storage unit 209 stores the new imaging result information received by the imaging result information reception unit 208 in the medical treatment DB 201.

Specific processing to be performed when the imaging result information reception unit 208 newly receives the input of imaging result information corresponding to the imaging ID “imaging 1”, imaging ID “imaging 2”, and imaging ID “imaging 3” will be described next.

In this case, in step 502, the imaging result information storage unit 209 stores the imaging IDs and imaging result information in the medical treatment DB 201 in association with each other. In the case shown in FIG. 3, the imaging ID “imaging 1” and the imaging result “failure” are stored in the medical treatment DB in association with each other. Likewise, the imaging ID “imaging 2” and the imaging result “failure” are stored in the medical treatment DB in association with each other, and the imaging ID “imaging 3” and the imaging result “success” are stored in the medical treatment DB in association with each other.

FIG. 6 is a flowchart showing re-imaging reception processing by the x-ray diagnostic imaging apparatus 101.

In step 601, if the re-imaging reception unit 210 receives the input of a new re-imaging designation (re-imaging reception processing), the process advances to step 602. In step 602, the re-imaging information storage unit 211 stores the new re-imaging information received by the re-imaging reception unit 210 in the medical treatment DB 201.

Specific processing to be performed when the re-imaging reception unit 210 newly receives the designation of re-imaging with respect to the imaging ID “imaging 1” and the imaging ID “imaging 2” will be subsequently described with reference to FIG. 3. In this case, in step 602, the re-imaging information storage unit 211 stores the imaging IDs and the re-imaging IDs in the medical treatment DB 201 in association with each other. In this case, as patient information and disease information, immediately preceding information can be used, and hence is copied and pasted.

In the case shown in FIG. 3, the imaging ID “imaging 1” and the re-imaging ID “imaging 2” are stored in the medical treatment DB in association with each other. Likewise, the imaging ID “imaging 2” and the re-imaging ID “imaging 3” are stored in the medical treatment DB in association with each other.

As described above, if pieces of past failure imaging information include imaging information similar to the currently input imaging information, the x-ray diagnostic imaging apparatus 101 according to the first embodiment displays information indicating there is a possibility that imaging may fail and information indicating specific imaging conditions that turned the failure into a success.

This allows the operator of the x-ray diagnostic imaging apparatus 101 to know that the initial imaging information set by himself/herself is appropriate/inappropriate, before actually performing imaging, and can provide indices for proper imaging conditions with respect to the set imaging information including information associated with the body of a patient (age, sex, height, and weight) and information concerning a disease. This enables the operator to perform imaging upon referring to these pieces of information and making re-setting as recommended or correcting some parameters. As a consequence, it is possible to increase the possibility of preventing wasteful imaging.

### Second Embodiment

An x-ray diagnostic imaging apparatus 101 according to the second embodiment will be described next. The x-ray diagnostic imaging apparatus 101 according to the second embodiment extracts success imaging information in further consideration of the cause of a failure when imaging has failed. Note that differences between the x-ray diagnostic imaging apparatus according to the second embodiment and the x-ray diagnostic imaging apparatus according to the first embodiment will be described below.

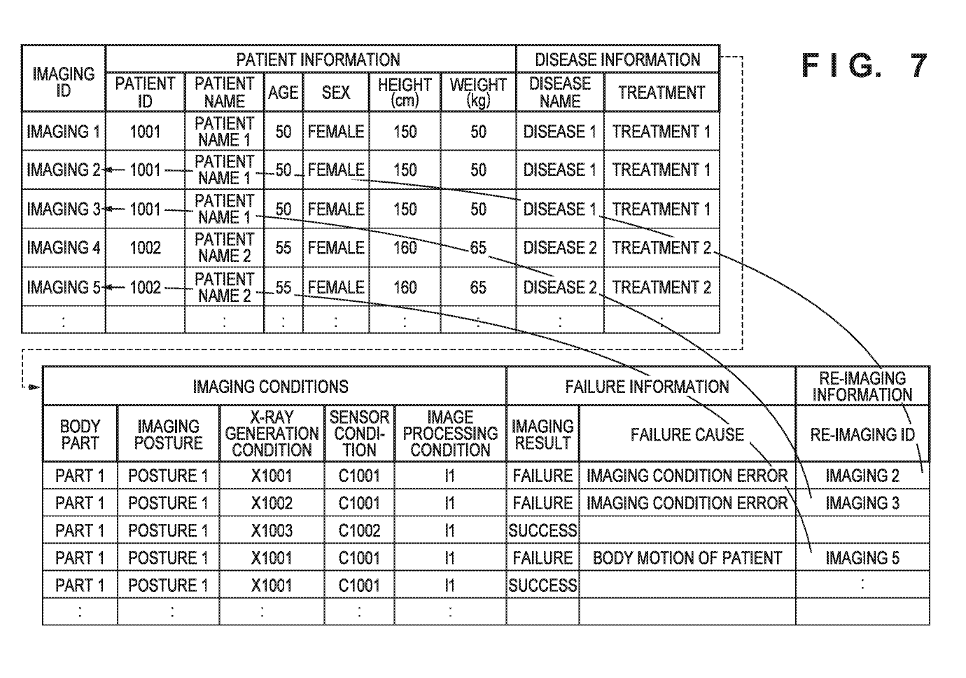

FIG. 7 is a view showing an example of the data arrangement of a medical treatment DB 201 according to the second embodiment. A difference from FIG. 3 is that fields for storing the causes of imaging failures are added. The cause of an imaging failure can be regarded as one of two factors, that is, a setting error of imaging conditions and the body motion of a patient. In the second embodiment, therefore, when displaying an obtained image on a display unit 107, a GUI for the evaluation is used to make the user select a success or failure and further select the cause of the failure if he/she selects it.

An imaging result information reception unit 208 according to the second embodiment, therefore, receives an imaging ID, an imaging result, and the cause of a failure if imaging has failed, which are input by the operator. An imaging result information storage unit 209 stores the imaging ID, the imaging result, and the cause of the failure if imaging has failed, which have been received by the imaging result information reception unit 208, in the medical treatment DB 201 in association with each other.

A failure imaging information extraction unit 204 according to the second embodiment then searches failure imaging information each corresponding to the imaging failure cause “imaging condition error” for failure imaging information exhibiting the minimum distance equal to or less than a threshold, while excluding imaging information with the imaging failure cause “body motion of patient” from search targets.

Even if, therefore, imaging information newly received by an imaging information reception unit 202 completely matches that corresponding to the imaging ID “imaging 4” in FIG. 7, the imaging conditions are not regarded as inappropriate imaging conditions. Imaging can therefore be normally performed.

In addition, the failure imaging information extraction unit 204 according to the second embodiment extracts imaging information having “imaging condition error” as a failure cause, and hence displays the cause of the imaging error in step 407 in FIG. 4. This makes it possible to explicitly show to the operator that the possibility of a defect in the imaging conditions is high.

Note that according to the second embodiment, when “body motion of patient” is stored in the failure cause field, the failure imaging information extraction unit 204 does not regard the corresponding imaging information as failure imaging information. However, when displaying the cause of an imaging failure on the display screen, even if “body motion of patient” is stored in the failure cause field, the corresponding information may be handled as failure imaging information. This is because, when information similar to information indicating that an imaging failure has been caused by “body motion of patient” is displayed on the screen, it is possible to determine that there is no defect in set imaging conditions.

Note that the arrangement and processing of the x-ray diagnostic imaging apparatus 101 according to the second embodiment are the same as those of the x-ray diagnostic imaging apparatus according to the first embodiment except for the above.

As has been described above, the x-ray diagnostic imaging apparatus 101 according to the second embodiment extracts success imaging information in accordance with input imaging information, past failure imaging information, and success imaging information at the time of re-imaging, and displays a past failure cause and the success imaging information as recommended imaging information, together with a message indicating that the input imaging information is inappropriate.

This enables the x-ray diagnostic imaging apparatus 101 or the operator to perform x-ray imaging with appropriate imaging information, while preventing x-ray imaging with inappropriate imaging information, when newly performing x-ray imaging based on the recommended imaging information and the past failure cause displayed by the x-ray diagnostic imaging apparatus.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2014-259277, filed Dec. 22, 2014, which is hereby incorporated by reference herein in its entirety.

What is claimed is:

1. A diagnostic imaging apparatus including an x-ray imaging unit, the apparatus comprising:
a reception unit configured to receive imaging information constituted by parameters concerning a target person and parameters concerning conditions for obtaining an x-ray image by making the x-ray imaging unit perform imaging;
a storage unit configured to store an evaluation result concerning an x-ray image obtained by making the x-ray imaging unit perform imaging in accordance with the imaging information in association with the imaging information;
a first searching unit configured to search, when the reception unit receives new imaging information for newly performing imaging, past imaging information stored in the storage unit for imaging information which has a similarity degree not less than a preset threshold relative to the new imaging information and is assigned with an evaluation result indicating an imaging failure; and
a second searching unit configured to search imaging information corrected with respect to the imaging information found by the first searching unit for imaging information assigned with the evaluation result indicating an imaging success as recommended imaging information in place of the new imaging information.

2. The apparatus according to claim 1, further comprising a display unit configured to display information indicating a possibility that imaging may fail with the new imaging information, together with the imaging information obtained by searching by the second searching unit, when the first searching unit has succeeded in searching for imaging information assigned with the evaluation result indicating an imaging failure and the second searching unit has succeeded in searching for imaging information assigned with the evaluation result indicating an imaging success.

3. The apparatus according to claim 2, wherein the display unit displays parameters, in the imaging information obtained by searching by the second searching unit, which concern conditions for obtaining an x-ray image by making the x-ray imaging unit perform imaging.

4. The apparatus according to claim 1, wherein the first searching unit regards the imaging information as a multidimensional coordinate space indicated by the number of parameters constituting the imaging information and searches for imaging information whose distance from a coordinate position indicated by the new imaging information is not more than a preset threshold and which is assigned with an evaluation result indicating an imaging failure.

5. The apparatus according to claim 1, wherein the parameters concerning the target person include an age, a sex, a height, and a weight of a patient, and
parameters setting conditions for obtaining an x-ray image by making the x-ray imaging unit perform imaging include a body part to be imaged, an imaging posture, an x-ray generation condition, and an image processing condition.

6. The apparatus according to claim 1, wherein the storage unit further stores information indicating an imaging failure cause with respect to each imaging information, and
the first searching unit narrows down search targets in accordance with the imaging failure cause.

7. A control method for a diagnostic imaging apparatus including an x-ray imaging unit and a storage unit configured to store an evaluation result concerning an x-ray image obtained by making the x-ray imaging unit perform imaging in accordance with imaging information constituted by parameters concerning a target person and parameters concerning conditions for obtaining an x-ray image by making the x-ray imaging unit perform imaging in association with the imaging information, the method comprising:
receiving imaging information constituted by parameters concerning a target person and parameters concerning conditions for obtaining an x-ray image by making the x-ray imaging unit perform imaging;
performing a first search to search, when new imaging information for newly performing imaging is received in the receiving, past imaging information stored in the storage unit for imaging information which has a similarity degree not less than a preset threshold relative to the new imaging information and is assigned with an evaluation result indicating an imaging failure; and
performing a second search to search imaging information corrected with respect to the imaging information found in the performing the first search for imaging information assigned with the evaluation result indicating an imaging success as recommended imaging information in place of the new imaging information.

8. The method according to claim 7, further comprising displaying information indicating a possibility that imaging may fail with the new imaging information, together with the imaging information obtained in the performing the second search, when the performing the first search has succeeded in searching for imaging information assigned with the evaluation result indicating an imaging failure and the performing the second search has succeeded in searching for imaging information assigned with the evaluation result indicating an imaging success.

9. The method according to claim 8, wherein in the displaying, parameters, in the imaging information obtained in the performing the second search, which concern conditions for obtaining an x-ray image by making the x-ray imaging unit perform imaging are displayed.

10. The method according to claim 7, wherein in the performing the first search, the imaging information is regarded as a multidimensional coordinate space indicated by the number of parameters constituting the imaging information and imaging information whose distance from a coordinate position indicated by the new imaging information is not more than a preset threshold and which is assigned with an evaluation result indicating an imaging failure is searched out.

11. The method according to claim 7, wherein the parameters concerning the target person include an age, a sex, a height, and a weight of a patient, and
parameters setting conditions for obtaining an x-ray image by making the x-ray imaging unit perform imaging include a body part to be imaged, an imaging posture, an x-ray generation condition, and an image processing condition.

12. The method according to claim 7, wherein the storage unit narrows down imaging targets in accordance with an imaging failure cause with respect each imaging information.

13. A non-transitory computer-readable storage medium storing a program which causes a computer, which has an x-ray imaging unit and a storage unit configured to store an evaluation result concerning an x-ray image obtained by making the x-ray imaging unit perform imaging in accordance with imaging information constituted by parameters concerning a target person and parameters concerning conditions for obtaining an x-ray image by making the x-ray imaging unit perform imaging in association with the imaging information, to execute steps of a method, where the method comprising:
receiving imaging information constituted by parameters concerning a target person and parameters concerning conditions for obtaining an x-ray image by making the x-ray imaging unit perform imaging;
performing a first search to search, when new imaging information for newly performing imaging is received in the receiving, past imaging information stored in the storage unit for imaging information which has a similarity degree not less than a preset threshold relative to the new imaging information and is assigned with an evaluation result indicating an imaging failure; and
performing a second search to search imaging information corrected with respect to the imaging information found in the performing the first search for imaging information assigned with the evaluation result indicating an imaging success as recommended imaging information in place of the new imaging information.
